(19) United States
(12) Patent Application Publication
(10) Pub. No.: US 2013/0018178 A1
(43) Pub. Date: Jan. 17, 2013

(54) METHOD FOR THE PURIFICATION OF HBHA

(71) Applicants: Aurelie Laronze, Grenoble (FR); Nicolas Mouz, Saint Paul De Varces (FR); Joana Pech, Saint Egreve (FR)

(72) Inventors: Aurelie Laronze, Grenoble (FR); Nicolas Mouz, Saint Paul De Varces (FR); Joana Pech, Saint Egreve (FR)

(73) Assignee: PX THERAPEUTICS, Grenoble (FR)

(21) Appl. No.: 13/379,976

(22) PCT Filed: Jun. 22, 2010
(86) PCT No.: PCT/EP10/58821

§ 371 Date: Dec. 21, 2011

(30) Foreign Application Priority Data
Jun. 22, 2009 (EP) 09163355.2

(51) Int. Cl. C07K 1/36; C07K 1/18; C07K 14/35

(52) U.S. Cl. 530/396

(57) ABSTRACT

The present invention concerns a new method for the purification of Heparin Binding Hemagglutinin (HBHA) robust and reproducible at an industrial scale. The purification is made from HBHA-producing microorganisms extracted in presence of a detergent, a solvent or a chaotropic agent, followed by a first elution on ion exchange resins. In a preferred embodiment, the elution on ion exchange resins is followed by a second chromatography on mixed mode sorbents.

The present invention concerns a new method for the purification of Heparin Binding Hemagglutinin (HBHA) robust and reproducible at an industrial scale. The purification is made from HBHA-producing microorganisms extracted in presence of a detergent, a solvent or a chaotropic agent, followed by a first elution on ion exchange resins. In a preferred embodiment, the elution on ion exchange resins is followed by a second chromatography on mixed mode sorbents.

The invention also concerns the purified HBHA obtained by said purification method and its use in therapy, particularly as vaccine for the prevention of tuberculosis and in a diagnostic method for in vivo or in vitro detection and differentiation of mammals, including humans, susceptible to be infected by Mycobacterium, particularly Mycobacterium tuberculosis, for active or latent infections.

### BACKGROUND OF THE INVENTION

Tuberculosis remains a major public health problem even in the 21st century, as it still is the most common cause of infectious disease-related mortality worldwide, with 1.6 million deaths globally recorded in 2005 by the World Health Organization, and more than 8.8 million new infections. As a result, approximately 2 billion individuals are estimated to be latently infected with the causative agent, Mycobacterium tuberculosis. In addition to being a threat to human health, mycobacterial diseases also have a serious economical impact because of their importance in veterinary medicine. Other mycobacterial species, such as the members of the Mycobacterium avium/intracellulare complex now are recognized as frequent opportunistic agents infecting immunocompromised individuals. It is clear that the development of new drugs, improved diagnostics, and vaccines is urgently needed. The detection of latent tuberculosis infection is a major component of tuberculosis control strategies.

Currently, there is no immunological test with satisfactory levels of sensitivity and specificity for the diagnosis of tuberculosis and the efficacy of the Bacillus Calmette-Guérin (BCG) vaccine in protection against Mycobacterium tuberculosis is variable.

The Heparin Binding Hemagglutinin (HBHA) is a 28-kDa, methylated, surface-exposed protein of Mycobacterium tuberculosis that mediates the interaction of the tubercle bacilli with the host, acting as an adhesin for nonphagocytic cells.

Several experimental findings have implicated HBHA in the systemic extrapulmonary dissemination of Mycobacterium tuberculosis [1], a major step in the development of the active form of the disease. This protein binds to sulfated glycoconjugates at the surface of epithelial cells via its C-terminal heparin-binding domain composed of several lysine-rich repetitions. It also promotes bacterial aggregation, presumably via specific coiled-coil interactions involving its N-terminal moiety [2].

Moreover, immunological studies have showed that HBHA provides high level of protection against Mycobacterium tuberculosis challenge in mice and guinea pig. Protective immunity induced by methylated HBHA is comparable to the one provided by vaccination with BCG. Therefore, HBHA protein could be a promising new vaccine candidate to prevent the development of tuberculosis and also a promising antigen in diagnosis of latent tuberculosis.

KR 20080070262 describes production of HBHA with recombinant microorganisms and the importance of the protein methylation pattern on the immune response. The protein, methylated or not, is produced in very low quantities and no extraction/isolation/purification is disclosed that would allow to produce HBHA at an industrial scale.

WO 03/044048 discloses the purification of an enzyme involved in methylation of HBHA. No extraction/isolation/purification of HBHA is disclosed that would allow producing HBHA at an industrial scale.

Currently, procedures for purifying HBHA from Mycobacterium tuberculosis or Mycobacterium bovis BCG extracts disclosed in the art, including WO 97/444463 and publications by Menozzi et al [3], Pethe et al [4] and Masungi et al [5], using heparin binding affinity cannot be reproduced at an industrial scale, all the more so because the currently available HBHA can only be extracted with a low associated purity, needing additional purification steps with laboratory techniques such as reverse-phase high-performance liquid chromatography which is not scalable for a large scale industrial production. Moreover affinity chromatography is expensive and has shorter life span than hydrophobic resins or ion exchange resins. There is a need for a robust and reproducible method for the purification of HBHA from a biomass of HBHA-producing microorganism at an industrial scale with high yields.

### BRIEF DESCRIPTION OF THE INVENTION

Therefore, the invention provides for a method for the purification of Heparin Binding Hemagglutinin (HBHA) comprising the steps of:
a) providing a medium comprising HBHA extracted from HBHA-producing microorganisms and a detergent, a solvent or a chaotropic agent
b) isolating HBHA from the said medium by chromatography on a ion exchange resin.

In a preferred embodiment, the isolated HBHA is further purified by a mixed mode chromatography (step c).

Indeed, the isolated and/or purified HBHA may be further processed for its use in therapy or in a diagnostic method.

In a particular embodiment of the invention, the method comprises the steps of:
a′) extracting HBHA from a HBHA-producing microorganism in presence of a detergent, a solvent or a chaotropic agent to provide a medium comprising the extracted HBHA
b) isolating HBHA from the said medium by chromatography on an ion exchange resin, and, optionally
c) purifying the isolated HBHA by/using a mixed mode chromatography.

The method of the invention may also comprise comprises a further step of:
d) polishing the purified isolated HBHA by size exclusion chromatography.

The invention also comprises a method for the purification of Heparin Binding Hemagglutinin (HBHA) comprising the steps of:
b) isolating HBHA from a medium comprising HBHA extracted from HBHA-producing microorganisms by chromatography on a ion exchange resin
c) purifying the isolated HBHA by chromatography on a mixed-mode sorbent, and, optionally
d) polishing the purified isolated HBHA by size exclusion chromatography.

The HBHA-producing microorganisms are selected among the group consisting of naturally HBHA-producing microorganisms and recombinant microorganisms transformed for producing HBHA and mixtures thereof.

The medium comprising HBHA extracted from the HBHA-producing microorganisms is advantageously obtained by extracting HBHA-producing microorganisms in presence of a detergent, a solvent or a chaotropic agent and the HBHA-producing microorganisms are preferably treated by lyses of the cells prior extraction, the cells being obtained from a culture medium (biomass).

The present invention concerns also a purified HBHA obtained by the method of the invention for its use in therapy. It also concerns a pharmaceutical composition, particularly a vaccine composition comprising a purified HBHA obtained by the method of the invention and a diagnostic test or kit for in vivo or in vitro detection and differentiation of mammals susceptible to be infected by Mycobacterium tuberculosis, for active or latent infection, comprising a purified HBHA obtained by the method the invention.

### DETAILED DESCRIPTION OF THE INVENTION

In the method of the invention, HBHA comprises any HBHA proteins and their variants, more particularly HBHA proteins having antigenic properties.

These HBHA proteins may be obtained from naturally HBHA-producing microorganisms particularly selected among the group of Mycobacterium or from recombinant microorganisms transformed in order to produce HBHA comprising a heterologous nucleic acid coding for HBHA, under control of regulatory elements functional in said microorganisms.

HBHA proteins obtained from naturally HBHA-producing microorganisms generally comprise post translational modifications and, particularly, methylation of the lysine residues. HBHA obtained from recombinant microorganisms may be variants of the known HBHAs where modifications have been introduced in the HBHA sequence, including substitutions, additions and deletions of microorganisms.

These HBHA proteins and potential variants are known in the art and disclosed particularly in WO 97/44463 and WO 2006/003029 which documents are incorporated herein by reference. From the teaching of these documents, the one of ordinary skill in the art understands the importance of the C-terminal fragment in the antigenic properties of HBHA, particularly for its use in vaccines and in diagnostic tests. Attention is drawn to the C-terminal fragment disclosed as SEQ ID NO 1 in WO 2006/003029 and the fragment disclosed on page 21 of WO 97/44463, line 13.

A preferred recombinant microorganism transformed for producing HBHA is Escherichia coli. However, other microorganisms known for being used in recombinant protein production such as Mycobacterium smegmatis, Lactococcus lactis, Pichia pastoris may also be used for the method of the invention.

In a preferred embodiment of the invention, the HBHA-producing microorganism is a non-pathogenic Mycobacterium, more preferably Mycobacterium bovis and Mycobacterium smegmatis. Mycobacterium bovis is certainly widely known in the art, more particularly the Mycobacterium bovis BCG strain. Such strain may be obtained from the cell culture collection (e.g. Mycobacterium bovis Karlson and Lessel TMC 1011 [BCG Pasteur] from Trudeau Mycobacterial Culture Collection, ATCC®number 35734™, Mycobacterium bovis Karlson and Lessel BCG, Copenhagen [H], ATCC® number 27290™.

Cell lyses are obtained from culture batches of HBHA-producing microorganisms. The one of ordinary skill in the art knows the numerous ways to culture microorganisms and produce a biomass consisting of these microorganisms.

The biomass is treated with usual techniques to obtain cell lyses, particularly in the presence of a detergent, a solvent or chaotropic agents. These techniques are standard techniques including mechanical shear, osmotic choc and enzymatic treatment. High-pressure cell disruption is a preferred technique for the production at an industrial scale.

Either detergents, solvents or chaotropic agents are used to prepare a medium comprising cell lyses from HBHA-producing microorganisms. Said detergent allows improved solubilisation of HBHA proteins. In a preferred embodiment, said detergent is selected among anionic surfactants such as sodium dodecyl sulfate, nonionic surfactants such as, for example, Triton® X-100 ((C₁₄H₂₂O(C.sub.₂H₄O)ₙ)) or Tween®-20, Tween®-80 (polyoxyethylene sorbitan monolaurate) (available from the Sigma-Aldrich Company of St Louis, Mo.), or zwitterionic detergents such as, for example, 3-[(3-cholamidopropyl) dimethylammonio]-1-propanesulfonate (CHAPS).

Detergents are used in a preferred embodiment and particularly Tween®-20 is a preferred detergent, particularly due to its compatibility with cGMP standards of production. In a preferred embodiment, solvents are selected among ethanol, isopropanol and acetonitrile and chaotropic agents among urea, guanidine or Hofineister ions.

The one of ordinary skill in the art will be able to determine the amount of detergent, solvent or chaotropic agent in the medium in order to extract the highest amount of available HBHA proteins. Preferably, the medium comprises from 0.001 to 20% of detergent, solvent or chaotropic agent, more preferably from 0.1 to 1%.

After cell disruption in presence of a detergent, a solvent or a chaotropic agent the lysate may be treated according to standard procedures known by the one of ordinary skill in the art to eliminate some by products and/or cells extracts and keep in the medium the HBHA previously extracted. Such procedures include clarification by either normal flow filtration, tangential flow filtration or centrifugation step under usual conditions.

The HBHA extracted in the medium comprising a detergent, a solvent or a chaotropic agent is first purified by chromatography on cation or anion exchange resins. Indeed, the biochemical properties of the protein can be easily modified by substitution of C-terminal lysine residues. HBHA are known to have different isoelectric points (pI) either from cationic forms having net positive charges to anionic forms having net negative charges, mainly because of differences in their C-terminus domain.

The one of ordinary skill in the art will determine the isoelectric point of the HBHA to be purified, either by theory according to usual calculation methods such as disclosed in Sillero, A. and Ribeiro, J. M., Isoelectric points of proteins: theoretical determination. Anal. Biochem. v179. 319-325 [6] or by experimental methods subjecting the compound of interest to electrophoresis, such as isoelectric focalisation as disclosed in “Isoelectric focusing in immobilized pH gradients: principle, methodology and some applications” by Bjellqvist B, Ek K, Righetti P G, Gianazza E, Görg A, Westermeier R, Postel W. or using Henderson-Hasselbalch equation [7].

Ion exchange resins are known in the art and used in the method of the present invention according to the specifications of the manufacturers. A cation exchange resin refers to a solid phase which is negatively charged, and which has free cations for exchange with cations in an aqueous solution passed over or through the solid phase. Any negatively charged ligand attached to the solid phase suitable to form the cation exchange resin can be used, e.g., a carboxylate, sulfonate and others commercially available cation exchange resins for examples, in particular but not limited to those having a sulfonate based group (e.g., MonoS, MiniS, Source 15S and 30S, SP Sepharose Fast Flow™, SP Sepharose High Performance from GE Healthcare, Toyopearl SP-650S and SP-650M from Tosoh, Macro-Prep High S from BioRad, Ceramic HyperD S, Trisacryl M and LS SP and Spherodex LS SP from Pall Technologies); a sulfoethyl based group (e.g., Fractogel SE, from EMD, Poros S-10 and S-20 from Applied Biosystems); a sulphopropyl based group (e.g., TSK Gel SP 5PW and SP-5PW-HR from Tosoh, Poros HS-20 and HS 50 from Applied Biosystems); a sulfoisobutyl based group (e.g., (Fractogel EMD SO.sub.3.sup.-from EMD); a sulfoxyethyl based group (e.g., SE52, SE53 and Express—Ion S from Whatman), a carboxymethyl based group (e.g., CM Sepharose Fast Flow from GE Healthcare, Hydrocell CM from Biochrom Labs Inc., Macro-Prep CM from BioRad, Ceramic HyperD CM, Trisacryl M CM, Trisacryl LS CM, from Pall Technologies, Matrx Cellufine C500 and C200 from Millipore, CM52, CM32, CM23 and Express—Ion C from Whatman, Toyopearl CM-650S, CM-650M and CM-650C from Tosoh); sulfonic and carboxylic acid based groups (e.g. BAKERBOND Carboxy-Sulfon from J. T. Baker); a carboxylic acid based group (e.g., WP CBX from J. T Baker, DOWEX MAC-3 from Dow Liquid Separations, Amberlite Weak Cation Exchangers, DOWEX Weak Cation Exchanger, and Diaion Weak Cation Exchangers from Sigma-Aldrich and Fractogel EMD COO— from EMD); a sulfonic acid based group (e.g., Hydrocell SP from Biochrom Labs Inc., DOWEX Fine Mesh Strong Acid Cation Resin from Dow Liquid Separations, UNOsphere S, WP Sulfonic from J. T. Baker, Sartobind S membrane from Sartorius, Amberlite Strong Cation Exchangers, DOWEX Strong Cation and Diaion Strong Cation Exchanger from Sigma-Aldrich); and a orthophosphate based group (e.g., P11 from Whatman).

Anion exchange resin refers to a solid phase which is positively charged, thus having one or more positively charged ligands attached thereto. Any positively charged ligand attached to the solid phase suitable to form the anionic exchange resin can be used, such as quaternary amino groups Commercially available anion exchange resins include DEAE cellulose, Poros PI 20, PI 50, HQ 10, HQ 20, HQ 50, D 50 from Applied Biosystems, Sartobind Q from Sartorius, MonoQ, MiniQ, Source 15Q and 30Q, Q, DEAE and ANX Sepharose Fast Flow, Q Sepharose high Performance, QAE SEPHADEX™ and FAST Q SEPHAROSE™ (GE Healthcare), WP PEI, WP DEAM, WP QUAT from J. T. Baker, Hydrocell DEAE and Hydrocell QA from Biochrom Labs Inc., UNOsphere Q, Macro-Prep DEAE and Macro-Prep High Q from Biorad, Ceramic HyperD Q, ceramic HyperD DEAE, Trisacryl M and LS DEAE, Spherodex LS DEAE, QMA Spherosil LS, QMA Spherosil M and Mustang Q from Pall Technologies, DOWEX Fine Mesh Strong Base Type I and Type II Anion Resins and DOWEX MONOSPHER E 77, weak base anion from Dow Liquid Separations, Intercept Q membrane, Matrex Cellufine A200, A500, Q500, and Q800, from Millipore, Fractogel EMD TMAE, Fractogel EMD DEAE and Fractogel EMD DMAE from EMD, Amberlite weak strong anion exchangers type I and II, DOWEX weak and strong anion exchangers type I and II, Diaion weak and strong anion exchangers type I and II, Duolite from Sigma-Aldrich, TSK gel Q and DEAE 5PW and 5PW-HR, Toyopearl SuperQ-6505, 650M and 650C, QAE-550C and 650S, DEAE-650M and 650C from Tosoh, QA52, DE23, DE32, DE51, DE52, DE53, Express-Ion D and Express-Ion Q from Whatman.

In a preferred embodiment, the HBHA is further purified on a mixed mode sorbents. Such resins are known in the art and used in the method of the invention according to the specifications of the manufacturers. These mixed-mode sorbents combine multiple interaction modes, in particular but not limited to, ionic, electrostatic and hydrophobic interactions which offer unique selectivity that may not be achievable by the sequentially use of single mode chromatography. Commercially available mixed-mode sorbents include, but are not limited to, BAKERBOND ABX™ (J. T. Baker; Phillipsburg, N.J.), ceramic hydroxyapatite type I and II and fluoride hydroxyapatite (BioRad; Hercules, Calif.) and HA-Ultrogel (Pall Corporation; East Hills, N.Y.), Capto MMC and Capto adhere (GE Healthcare) and MEP, HEA, PPA and MBI HyperCel (Pall Corporation; East Hills, N.Y.)

The product being isolated and purified may be further processed, particularly for its use in therapy and diagnostic tests. Said processes may include further purification in order to obtain a higher grade of purity, and/or usual treatment for conservation and formulation of proteins, such as diafiltration and/or freeze drying.

In a preferred embodiment, the HBHA is further purified by a “polishing” step of purification. Such “polishing” step is known to the person skilled in the art of protein purification, such as size exclusion chromatography. The technique also known as gel filtration chromatography is often reserved for the final “polishing” step of purification. In the method, molecules in an aqueous solution are separated based on their size (or hydrodynamic volume) through gel medium—usually polyacrylamide, dextran or agarose and filtered under low pressure. Commercially available gel filtration resins include, but are not limited to, Superdex75 (Superdex™ 75 Prep Grade resin, GE Healthcare).

With the method of the invention, one may obtain more than 60% of HBHA proteins available in the biomass, with a purity of more than 80%. This may be compared with the standard procedure disclosed in the art where only a low purity associated HBHA is available in the biomass obtained.

When the starting material is the Mycobacterium bovis BCG strain, the product is obtained with the following characteristics:
the final buffer: 1× Phosphate Buffered Saline (PBS);
a purity grade of at least 83.7%, preferably at least 98%, determined by reverse-phase high performance liquid chromatography. The summary of contaminant removal using the purification scheme disclosed in the invention is represented by FIGS. 1 and 10;
N-terminal sequencing based on Edman degradation matches the sequence of the N-terminal of HBHA, from the second residue (AENSNIDDI);
The process productivity was about 1 mg to about 5 mg purified HBHA from 20 g of Mycobacterium bovis BCG.

The purified HBHA obtained by the method of the invention may be used in therapy, particularly in a pharmaceutical composition, including vaccine composition, for mammals, including humans.

Such vaccine preparations are well known in the art and comprise suitable pharmaceutically acceptable carriers, such as excipients which facilitate the immunogenic effect of the protein extracted and purified by the method of the invention. Such carriers are preferably suitable adjuvants that release an immunogen in vivo over a prolonged period as compared to administration of an unbound immunogen. Non-limiting examples of such adjuvants comprise an aluminium, calcium or salts thereof, such as aluminium sulphate, aluminium phosphate, calcium phosphate, aluminium potassium sulphate, and aluminium hydroxyphosphate sulfate or aluminium hydroxide. Other non-limiting examples of preferred carriers are those that target macrophages and/or activate them, such as liposomes or proteoliposomes, or the virus-like particles, such as virosomes, consisting of empty viral envelopes that can be loaded to carry antigens.

The purified HBHA obtained by the method of the invention may also be used in diagnostic tests for in vivo or in vitro detection and differentiation of mammals likely to be infected by Mycobacterium tuberculosis, for active or latent infection, comprising a purified HBHA obtained by the method of the invention. Diagnostic tests and kits comprising the purified HBHA are also part of the invention.

Such methods, tests and kits are well known in the art, and disclosed in WO 97/44463 and WO 2006/00309, which content is incorporated herein by reference.

### FIGURES

FIG. 1 represents the overview of contaminant removal using the suggested purification scheme.

FIG. 2 represents the effect of detergents on native HBHA solubilisation.

FIG. 3 shows the chromatographic profile of the cation exchange chromatography (e.g. SP Sepharose Fast Flow from GE Healthcare) described in the example 2.

FIG. 4 represents the capture of native HBHA produced by Mycobacterium bovis onto the cation exchange chromatography (e.g. SP Sepharose Fast Flow from GE Healthcare).

FIG. 5 shows the chromatographic profile of the anion exchange chromatography (e.g. Q Sepharose high performance from Sigma) described in the example 2.

FIG. 6 represents the capture of recombinant HBHA produced by Escherichia coli onto the anion exchange chromatography (e.g. Q Sepharose high performance from Sigma).

FIG. 7 shows the chromatographic profile of the anion exchange chromatography (e.g. MEP Hypercel from Pall Life Sciences) described in the example 3.

FIG. 8 represents the purification of HBHA protein by a mixed mode chromatography (e.g. MEP Hypercel from Pall Life Sciences).

FIG. 9 represents HBHA protein loaded on 4-12% Bis-Tris SDS-PAGE gel stained by Silver nitrate. M: Marker.

FIG. 10 represents the purity of the HBHA protein after polishing analyzed by RP-HPLC (Column: C4 BEH300 4.6×150 mm).

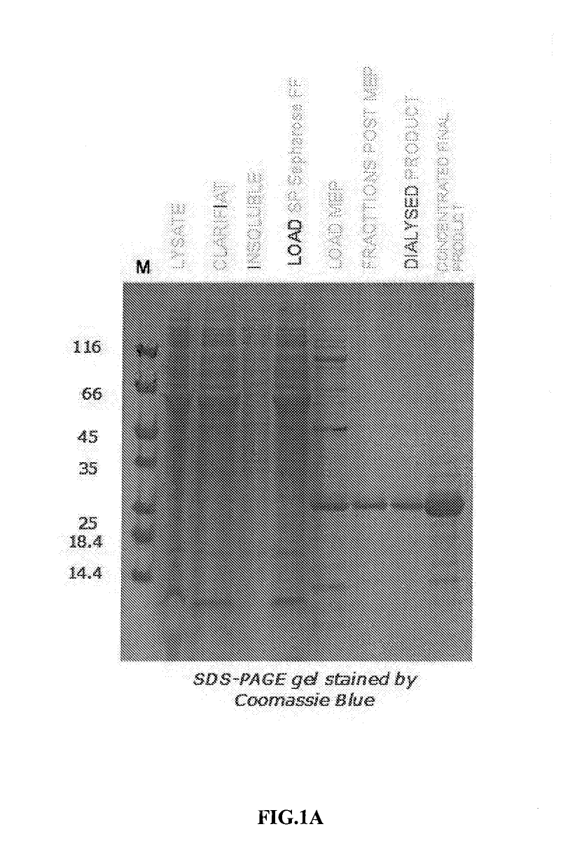

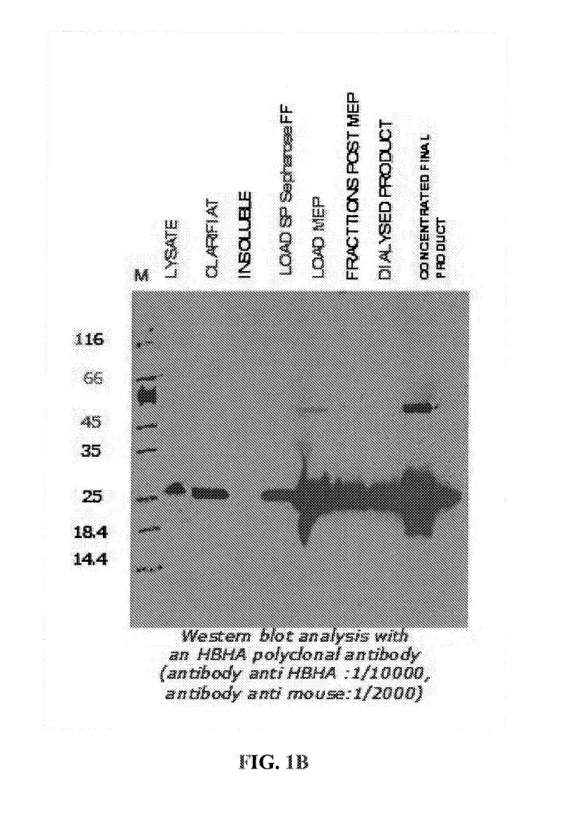

### EXAMPLES

The invention will be further described and detailed by the following examples, which are intended to be purely instance and illustrate the method of the invention, and should not be considered as limiting the invention in any way.

### Example 1

### Effect of Detergents on HBHA Solubilisation

The purpose of this example was to demonstrate the effect of detergents on the extraction and the solubilisation of native HBHA from Mycobacterium bovis BCG.

To inactivate the whole Mycobacterium bovis strain, the pellet was heated at 80° C. for 30 minutes. After inactivation, the pellet was resuspended in lysis buffer: 1×PBS in presence of detergent (nonionic surfactants such as 1% Triton®X-100 ((C₁₄H₂₂O(C.sub.2H₄O)ₙ)) and 0.05% Tween®-80 (polyoxyethylene sorbitan monolaurate) or zwitterionic detergents such as 0.2% and 1% CHAPS (3-[(3-cholamidopropyl)dimethylammonio]-1-propanesulfonate). Cell lysis was performed by sonication for 7 minutes, 3 times in a row. Soluble and insoluble proteins were separated by centrifugation at 13,600 g for 20 minutes.

Sodium dodecyl sulfate-polyacrylamide gel electrophoresis (SDS-PAGE) was performed as described by Laemmli, using a 4% stacking and 12% separating gel (Biorad); then the gel was stained with Coomassie blue or the proteins were transferred from the gel to a nitrocellulose membrane. The slides were incubated with anti-HBHA mouse sera at a 1:10000 dilution for 1 h at room temperature, washed extensively with 1×PBS, 0.3% Tween®-80, and incubated with peroxidase-conjugated anti-mouse IgG at a 1:2000 dilution for 1 h. Western blot was developed using the ECL Plus Western Blotting Detection Reagents (Fischer scientific) according to manufacturer instructions. Bands were observed exposing the membrane to autoradiography film for 10 minutes.

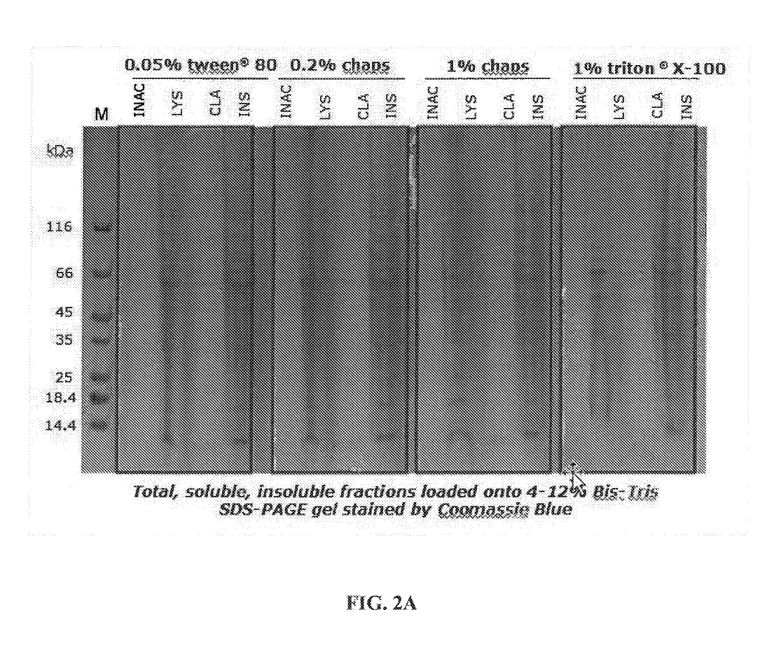

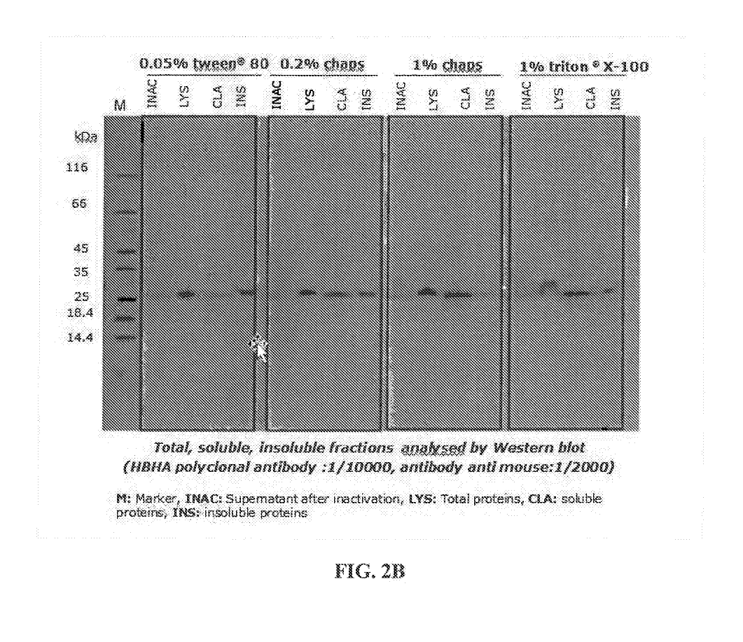

The efficacy of detergents on cell disruption and protein solubilisation from Mycobacterium bovis was assessed on the amount of HBHA soluble extracted in comparison with the insoluble fractions. According to the western blot analysis (FIG. 2), the zwitterionic detergents seems to be the most effective solubilisation buffers with respectively 50 and 80% of soluble HBHA for 0.2% and 1% CHAPS. Conversely, 0.05% Tween®-80 did not solubilise the protein of interest in the conditions of the assay.

### Example 2

### Purification of HBHA by Ion Exchange Chromatography

The present example describes the capture of HBHA protein using an ion exchange chromatography step to isolate HBHA from the crude extract.

HBHA are known to have different isoelectric points (pI) either from cationic forms having net positive charges to anionic forms having net negative charges, mainly because of differences in their C-terminus domain. Depending on the pI of HBHA protein, the crude extract can be purified either on cation exchange chromatography if the pI is basic or on anion exchange if the pI is acid.

Both native HBHA from Mycobacterium bovis (pI=9.17) and recombinant HBHA from Escherichia coli (pI=6.50) were chosen to illustrate this example.

Cation Exchange Chromatography of the Native HBHA Produced in Mycobacterium bovis

After extraction by high-pressure homogenization in presence of a detergent and clarification, the crude extract is loaded into a cation exchange chromatography media (e.g. SP Sepharose Fast Flow from GE Healthcare) equilibrated in PBS pH7.4. As the crude extract flows through the media, HBHA and other impurities are bound to the media. After binding, the media is washed with the equilibration buffer and with an additional washing step (e.g. PBS supplemented with 20 to 200 mM NaCl) in order to maximize the removal of contaminants. HBHA is then eluted with PBS supplemented with 100 to 500 mM NaCl, prior to regeneration of the media with a high salt concentration (e.g. PBS, 1M NaCl). Sodium dodecyl sulfate-polyacrylamide gel electrophoresis (SDS-PAGE) was performed as described by Laemmli by using a 4% stacking and 12% separating gel (Biorad); then the gel was stained with Coomassie blue or the proteins were transferred from the gel to a nitrocellulose membrane. The slides were incubated with anti-HBHA mouse sera at a 1:10000 dilution for 1 h at room temperature, washed extensively with 1×PBS, 0.3% Tween®-80, and incubated with peroxidase-conjugated anti-mouse IgG at a 1:2000 dilution for 1 h. Western blot was developed using the ECL Plus Western Blotting Detection Reagents (Fischer scientific) according to manufacturer instructions. Bands were observed exposing the membrane to autoradiography film for 10 minutes.

Anion Exchange Chromatography of the Recombinant HBHA Produced in Escherichia coli

After extraction by sonication and chemical lysis in presence of a detergent and clarification, the crude extract is loaded into an anion exchange chromatography media (e.g. Q Sepharose high performance from Sigma) equilibrated in 50 mM Tris pH8.0, 0 to 200 mM NaCl. As the crude extract flows through the media, HBHA and other impurities are bound to the media. After binding, the media is washed with the equilibration buffer. HBHA is then eluted and separated from impurities by a 10-30CV gradient from 0-300 mM NaCl to 1M NaCl, high salt concentration useful for medium regeneration. Sodium dodecyl sulfate-polyacrylamide gel electrophoresis (SDS-PAGE) was performed as described by Laemmli by using a 4% stacking and 12% separating gel (Biorad); then the gel was stained with Coomassie blue.

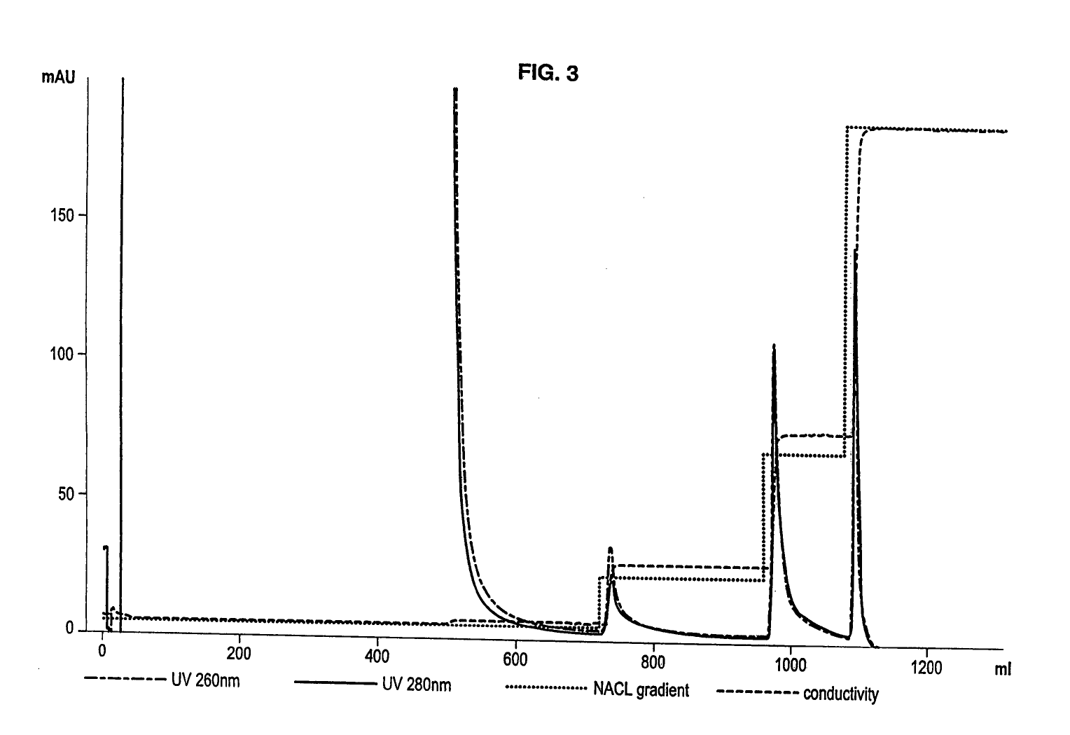

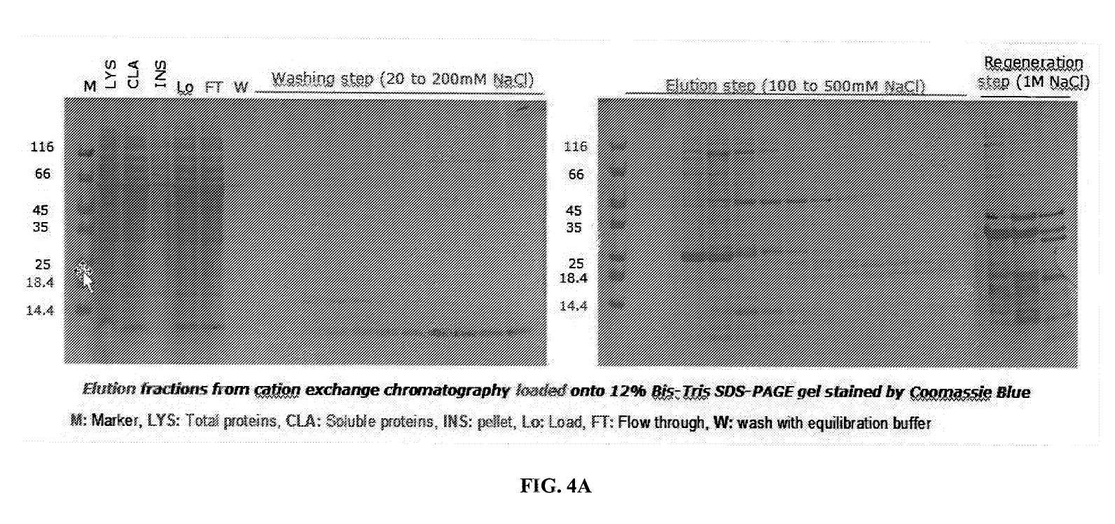

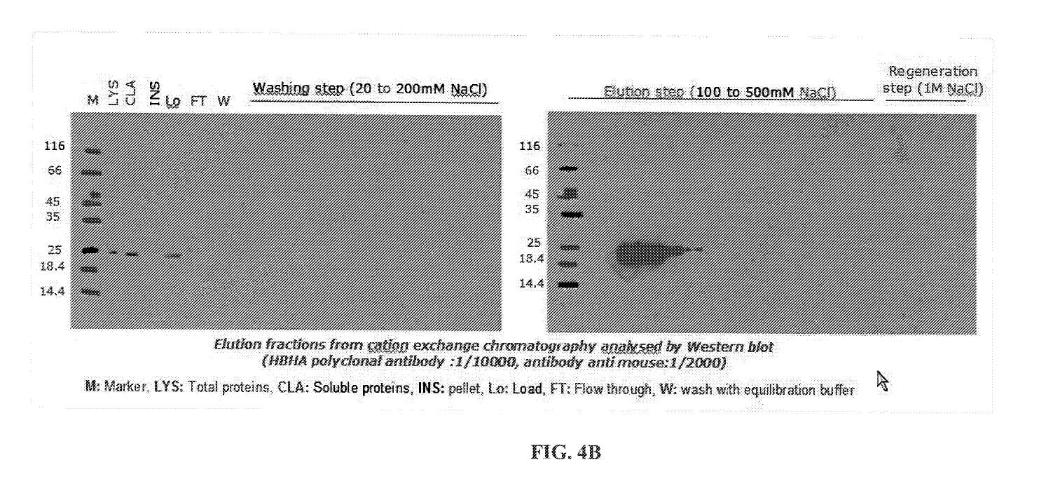

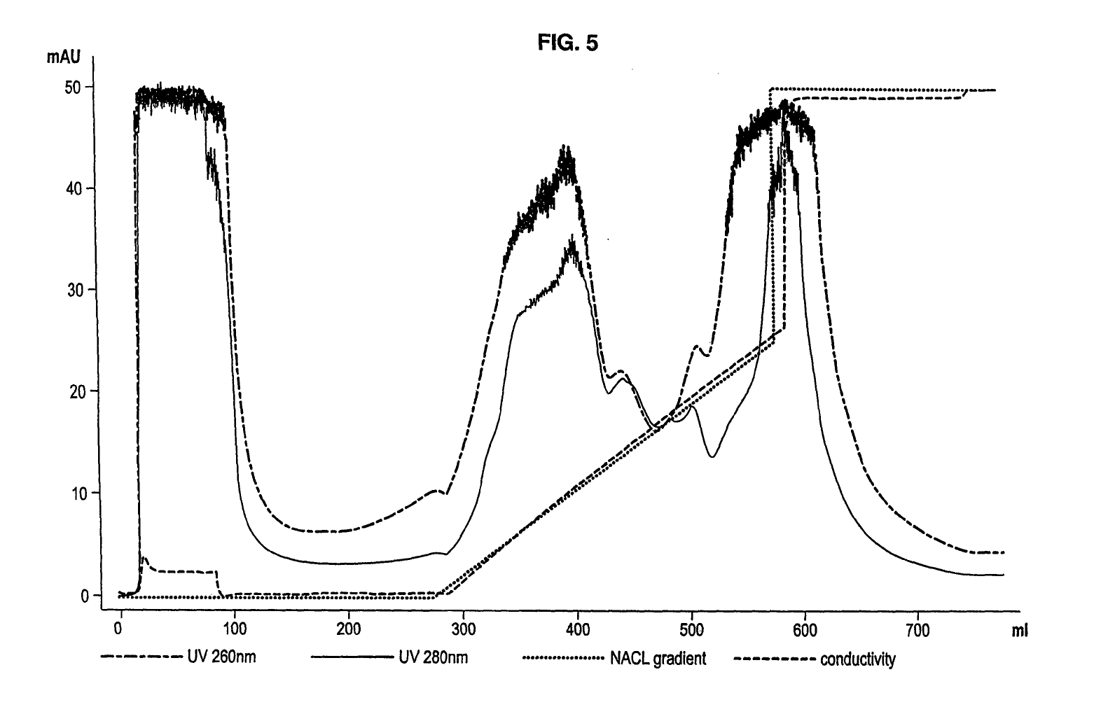

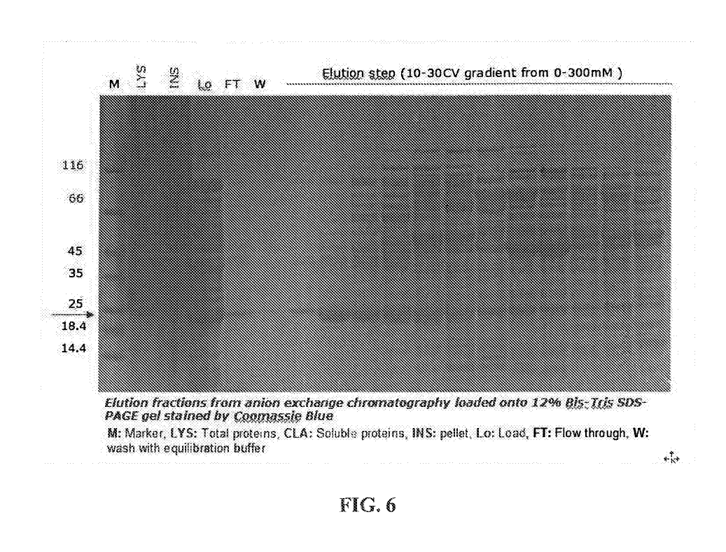

The Coomassie blue stained polyacrylamide gel and western blot analysis (FIGS. 3-4 for cation exchange chromatography and FIGS. 5-6 for anion exchange chromatography) showed that all the protein of interest was bound to the media. Indeed, no protein of interest was either present in the flow through or the washing step. Both native and recombinant HBHA were eluted with a concentration in salt between 100 and 500 mM NaCl. The ion exchange chromatography allows isolating HBHA protein from the crude extract with a purity grade superior to 25% and a recovery of 80%.

### Example 3

### Purification of HBHA by Mixed-Mode Chromatography

After purification by ion exchange chromatography, either by cation exchange or anion exchange, the pool containing HBHA is loaded onto a mixed-mode chromatography resin (e.g. MEP Hypercel from Pall Life Sciences) after equilibration of the medium into a buffer equivalent to the elution buffer of the ion exchange chromatography (e.g. 1×PBS with 100 to 500 mM NaCl). After binding of HBHA and other impurities onto the column, the medium is washed with the equilibration buffer and with an additional washing step (e.g. 50 mM sodium phosphate pH7.4 to 5.0 with 100 to 500 mM NaCl) in order to maximize removal of contaminants. HBHA is then eluted with 25 mM sodium acetate pH 6.0 to 4.0 supplemented with 0 to 200 mM NaCl, prior to regeneration of the media with a low pH (e.g. 25 mM citric acid pH3.0).

Sodium dodecyl sulfate-polyacrylamide gel electrophoresis (SDS-PAGE) was performed as described by Laemmli by using a 4% stacking and 12% separating gel (Biorad); then the gel was stained with Coomassie blue or the proteins were transferred from the gel to a nitrocellulose membrane. The slides were incubated with anti-HBHA mouse sera at a 1:10000 dilution for 1 h at room temperature, washed extensively with 1×PBS, 0.3% Tween®-80, and incubated with peroxidase-conjugated anti-mouse IgG at a 1:2000 dilution for 1 h. Western blot was developed using the ECL Plus Western Blotting Detection Reagents (Fischer scientific) according to manufacturer instructions. Bands were observed exposing the membrane to autoradiography film for 10 minutes.

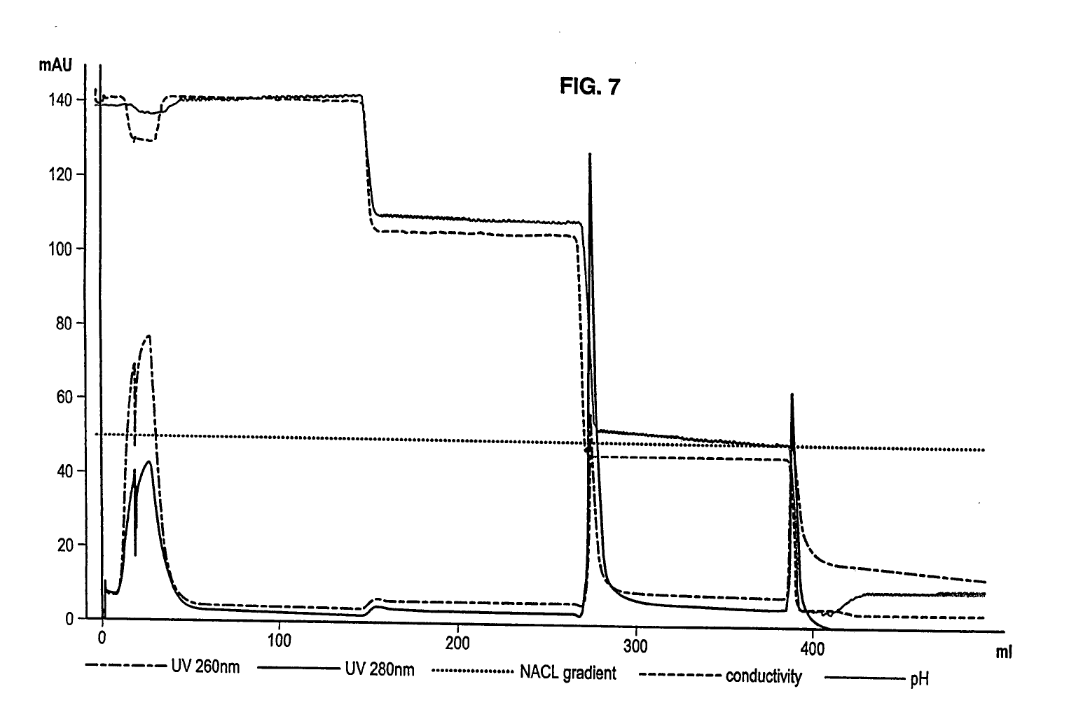

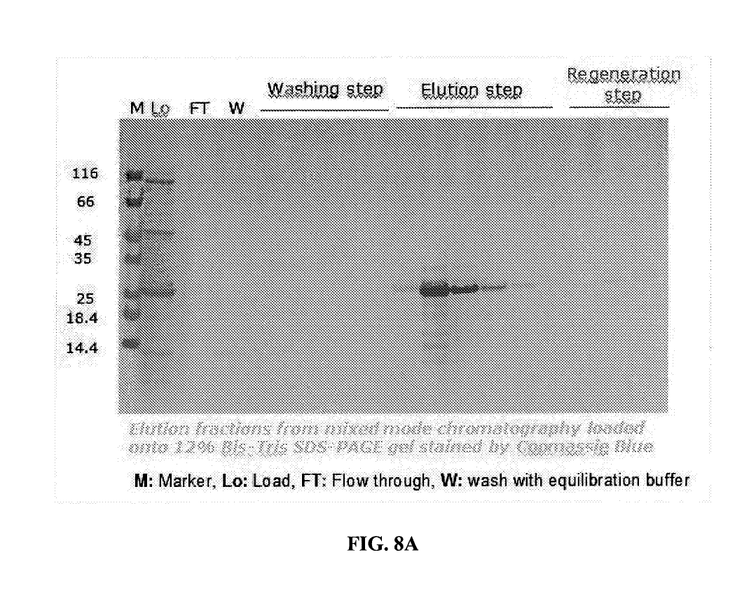

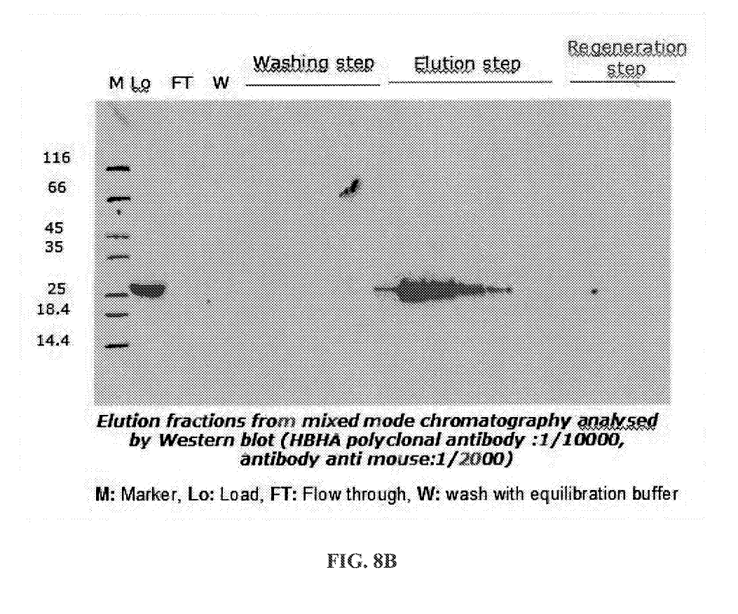

The mixed mode chromatography allows capturing all the protein interest. No HBHA protein was either in the flow trough or the washing step as reported by the coomassie blue stained and the western blot analysis (FIGS. 7-8). This purification step allows improving the purity from 25 to 80% with a high recovery.

### Example 4

### Purification of HBHA by Size-Exclusion Chromatography

The present example describes the final polishing step of HBHA purification using a size-exclusion chromatography.

The HBHA elution pool from mixed-mode chromatography is loaded onto a size-exclusion chromatography media (e.g. Superdex™ 75 Prep Grade resin, GE Healthcare) equilibrated in the working buffer 1×PBS pH7.4, 0.005% Tween®20. The HBHA protein goes through the spherical particles of the resin and is excluded regarding its oligomeric state. This polishing step allows to get a pure HBHA protein.

### Example 5

### Comparison of Industrial Scale and Research Grade HBHA Production Processes

| | Research grade protocol | Industrial scale process |
|---|---|---|
| Extraction | Extraction with detergent | Extraction with detergent |
| Capture | Heparin sepharose 6 Fast | SP Sepharose Fast Flow |
| chromatography | Flow (GE Healthcare) | (GE Healthcare) |
| HBHA captured on | 0.5 mg | 1 mg to 5 mg |
| first step | | |
| chromatography for | | |
| 10 g of biomass | | |

The process productivity after the capture chromatography was 10-fold higher with the industrial process defined by the method of the invention, in comparison with research grade procedures disclosed in the art.

### Example 6

### Process of the HBHA Purification

A process for the HBHA purification is performed with the following operating conditions:

1/HBHA extraction in presence of detergent (Tween®20)
2/Clarification by centrifugation
3/HBHA capture by cation exchange chromatography (SP Sepharose Fast Flow from GE healthcare)
4/HBHA purification by mixed-mode chromatography (MEP Hypercall from Pall Life Science)
5/Polishing by size-exclusion chromatography (Superdex™ 75 Prep Grade from GE healthcare)

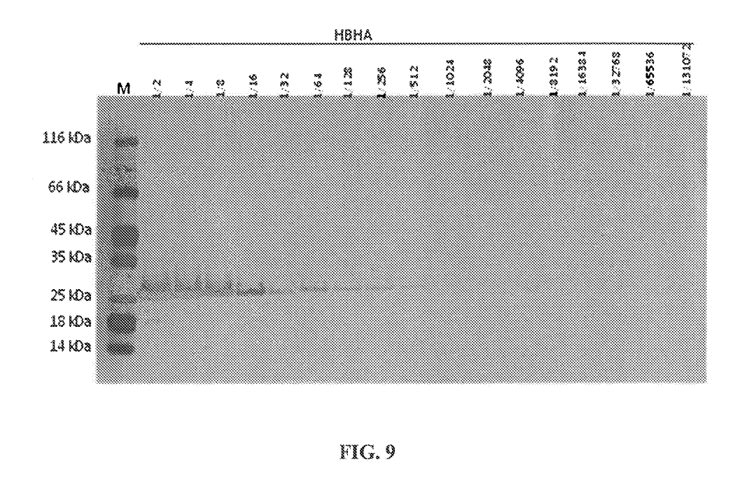

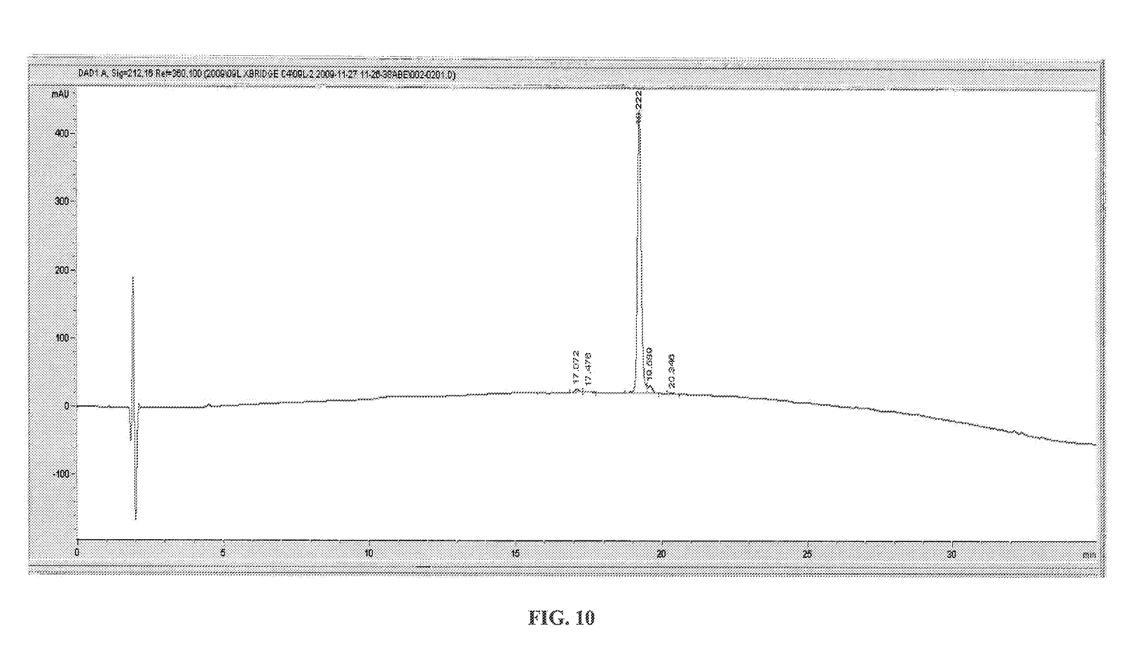

This process allows obtaining a HBHA protein with a high purity>99%. The purity of the HBHA protein is analyzed by RP-HPLC (Column: C4 BEH300 4.6×150 mm) (FIG. 10) and on SDS-PAGE gel stained by silver nitrate (FIG. 9).

### REFERENCES

[1]: Pethe, K., S. Alonso, F. Biet, G. Delogu, M. J. Brennan, C. Locht, and F. D. Menozzi. 2001. The heparin-binding haemagglutinin of M. tuberculosis is required for extrapulmonary dissemination. Nature 412:190-194
[2]: Delogu, G., and M. J. Brennan. 1999. Functional domains present in the mycobacterial hemagglutinin, HBHA. J. Bacteriol. 181:7464-7469
[3]: Menozzi, F. D., R. Bischoff, E. Fort, M. J. Brennan, and C. Locht. 1998. Molecular characterization of the mycobacterial heparin-binding hemagglutinin, a mycobacterial adhesin. Proc. Natl. Acad. Sci. USA 95:12625-12630.
[4]: Pethe K, Bifani P, Drobecq H, Sergheraert C, Debrie A S, Locht C, Menozzi F D. Mycobacterial heparin-binding hemagglutinin and laminin-binding protein share antigenic methyllysines that confer resistance to proteolysis. Proc Natl Acad Sci USA. 2002 Aug. 6; 99(16):10759-64. Epub 2002 Jul. 29.
[5]: Masungi, C., S. Temmerman, J. P. Van Vooren, A. Drowart, K. Pethe, F. D. Menozzi, C. Locht, and F. Mascart. 2002. Differential T and B cell responses against Mycobacterium tuberculosis heparin-binding hemagglutinin adhesin in infected healthy individuals and patients with tuberculosis. J. Infect. Dis. 185:513-520.
[6]: Sillero, A. and Ribeiro, J. M., Isoelectric points of proteins: theoretical determination. Anal. Biochem. v179.319-325.
[7]: Bjellqvist B, Ek K, Righetti P G, Gianazza E, Görg A, Westermeier R, Postel W. Isoelectric focusing in immobilized pH gradients: principle, methodology and some applications. J Biochem Biophys Methods. 1982 September; 6(4):317-39.

1. A method for purification of Heparin Binding Hemagglutinin (HBHA) comprising:
a) providing a medium comprising HBHA extracted from HBHA-producing microorganisms in presence of a detergent, a solvent and/or a chaotropic agent
b) isolating HBHA from the said medium by chromatography on a ion exchange resin.

2. A method for purification of Heparin Binding Hemagglutinin (HBHA) comprising:
a′) extracting HBHA from a HBHA-producing microorganism in presence of a detergent, a solvent and/or a chaotropic agent to provide a medium comprising the extracted HBHA
b) isolating HBHA from said medium by chromatography on an ion exchange resin.

3. The method of claim 1, wherein HBHA-producing microorganisms are at least one selected among from the group consisting of naturally HBHA-producing microorganisms and recombinant microorganisms transformed for producing HBHA.

4. The method of claim 3, wherein the naturally HBHA-producing microorganisms are selected from the group consisting of Mycobacterium.

5. The method of claim 4, wherein Mycobacterium is a non-pathogenic Mycobacterium, optionally comprising Mycobacterium bovis or Mycobacterium smegmatis.

6. The method of claim 3, wherein the recombinant microorganisms transformed for producing HBHA is a microorganism comprising a heterologous nucleic acid coding for HBHA, under control of regulatory elements functional in said microorganism, optionally selected from the group consisting of Escherichia coli, Mycobacterium smegmatis, Lactococcus lactis and Pichia pastoris.

7. The method of claim 1, wherein the detergent is at least one selected from the group consisting of anionic surfactants, nonionic surfactants and zwitterionic detergents.

8. The method of claim 1, wherein the ion exchange resin is a cation exchange resin and/or an anion exchange resin.

9. The method of claim 1, further comprising:
c) purifying isolated HBHA by chromatography on a mixed-mode sorbent.

10. The method of claim 9, wherein mixed mode sorbent chromatography is selected from the group consisting of HCIC resins.

11. The method of claim 1, further comprising:
d) polishing purified isolated HBHA by size exclusion chromatography.

12. A method for purification of Heparin Binding Hemagglutinin (HBHA) comprising:
a) providing a medium comprising HBHA extracted from HBHA-producing microorganisms in presence of a detergent, a solvent and/or a chaotropic agent
b) isolating HBHA from the said medium by chromatography on a ion exchange resin
c) purifying the isolated HBHA by chromatography on a mixed-mode sorbent, and
d) polishing the purified isolated HBHA by size exclusion chromatography.

13. A method for purification of Heparin Binding Hemagglutinin (HBHA) comprising:
b) isolating HBHA from a medium comprising HBHA extracted from HBHA-producing microorganisms by chromatography on a ion exchange resin
c) purifying isolated HBHA by chromatography on a mixed-mode sorbent, and/or optionally
d) polishing purified isolated HBHA by size exclusion chromatography.

14. The method of claim 1, wherein isolated and/or purified HBHA is processed for use in therapy.

15. (canceled)

16. A pharmaceutical composition comprising purified HBHA obtained by a method of claim 1.

17. A diagnostic test or kit for in vivo or in vitro detection and differentiation of mammals susceptible to be infected by Mycobacterium tuberculosis, for active or latent infection, comprising purified HBHA obtained by a method of claim 1.

18. The method of claim 2, wherein the HBHA-producing microorganisms are at least one selected from the group consisting of naturally HBHA-producing microorganisms and recombinant microorganisms transformed for producing HBHA.

19. The method of claim 18, wherein the naturally HBHA-producing microorganisms are selected among the group of Mycobacterium.

20. The method of claim 19, wherein the Mycobacterium is a non-pathogenic Mycobacterium, optionally comprising Mycobacterium bovis or Mycobacterium smegmatis.

21. The method of claim 20, wherein the recombinant microorganism transformed for producing HBHA, is a microorganism comprising a heterologous nucleic acid coding for HBHA, under control of regulatory elements functional in said microorganism, optionally selected from the group consisting of Escherichia coli, Mycobacterium smegmatis, Lactococcus lactis and Pichia pastoris.

22. The method of claim 18, wherein the detergent is selected from the group consisting of anionic surfactants, nonionic surfactants and zwitterionic detergents.

23. The method of claim 18, wherein the ion exchange resin is a cation exchange resin and/or an anion exchange resin.

24. The method of claim 18, comprising:
c) purifying the isolated HBHA by chromatography on a mixed-mode sorbent.

25. The method of claim 24, wherein mixed mode sorbent chromatography is selected among HCIC resins.

26. The method of claim 2, further comprising:
d) polishing purified isolated HBHA by size exclusion chromatography.

27. A pharmaceutical composition comprising purified HBHA obtained by a method of claim 12.

28. A pharmaceutical composition comprising purified HBHA obtained by a method of claim 13.

29. A pharmaceutical composition comprising purified HBHA obtained by a method of claim 18.
